(19) United States
(12) Patent Application Publication
(10) Pub. No.: US 2012/0309647 A1
(43) Pub. Date: Dec. 6, 2012

(54) PATTERNING AND CELLULAR CO-CULTURE

(75) Inventors: John M. Collins, West Chicago, IL (US); Saju R. Nettikadan, Hawthorn Woods, IL (US)

(73) Assignee: Nanolnk, Inc.

(21) Appl. No.: 13/483,915

(22) Filed: May 30, 2012

Related U.S. Application Data
(60) Provisional application No. 61/491,795, filed on May 31, 2011.

(51) Int. Cl. C40B 50/14; C40B 40/00; C40B 30/06; C12N 5/07

(52) U.S. Cl. 506/10; 506/30; 435/373; 506/13

(57) ABSTRACT

Cell co-culturing methods and arrays providing versatility and high resolution. A method comprising: providing at least one substrate; providing at least one first tip with at least one first cell-adhesion material disposed thereon, and at least one second tip with at least one second cell-adhesion material disposed thereon, wherein the first cell-adhesion material is different from the second cell-adhesion material; depositing the first cell-adhesion material from the first tip to the substrate to form at least one first deposit, and depositing the second cell-adhesion material from the second tip to the substrate to form at least one second deposit; and wherein the first and second deposits are capable of providing selective binding to at least one first cell so that the first cell selectively binds to the first deposit, and wherein the second deposit is capable of binding to at least one second cell.

### RELATED APPLICATIONS

This application claims priority to U.S. provisional application Ser. No. 61/491,795 filed May 31, 2011, which is hereby incorporated by reference in its entirety.

### BACKGROUND

Cell patterning has become a useful tool for probing many research questions and commercializing new products and methods. As a result many different tools have been developed to address this need. For example, patterning of proteins onto surfaces using microcontact printing has been previously described [1,2]. However, there are limitations to the patterning technique used by prior art, such as consistency of patterning [3]. Also, it can be relatively difficult to change pattern variables such as feature size and spacing. Moreover, it is difficult in most cases to print multiple proteins with precise control of size, spacing and leveling. A need exists to provide versatile, new patterning methodologies.

In addition, co-culture experiments have yielded valuable data related to the microenvironment necessary to induce stem or progenitor cell differentiation. For example, coculture experiments in which stem cells have been put into the cardiac microenvironment (i.e. coculture with primary cardiac myocytes) demonstrate that these cells are capable of differentiation into cardiac cells. Cardiac differentiation of cells such as endothelial progenitors [4,5] and bone marrow derived mesenchymal stem cells [6,7] after coculture with primary cardiac myocytes in vitro has been demonstrated. In fact, the ability of mesenchymal stem cells to differentiate into cardiac cells as demonstrated by coculture experiments has led to several clinical trials. However, the results from these trials have been limited possibly due to inconsistencies in the laboratory resulting from investigators using different cell populations as well as cell fusion or imaging artifacts [8]. Characterizing these cues might allow a better understanding of the role the microenvironment plays in stem cell function and thus the opportunity to control these factors in vivo and improve treatment of damaged tissue.

However, co-culturing can impose difficulties in the fabrication methods. For example, the cells should be kept alive throughout the processing. Also, it can be difficult to fabricate a patterned substrate for single-cell-based coculture, e.g., the coculture of at least two different single cells side-by-side.

Technical literature includes Kim et al., “Patterned Cocultures for Controlling Cell-Cell Interactions,” Chapter 4, 53-70, Micro and Nanoengineering of the Cell Microenvironment: Technologies and Applications, Artech House, (Eds. Khademhosseini et al.) (2008); Kaji et al., “Engineering Systems for the Generation of Patterned Co-cultures for Controlling Cell-Cell Interactions,” Biochimica et Biophysica Acta, 1810 (2011) 239-250; Khademhosseini et al., “Micro- and Nanoscale Control of Cellular Environment for Tissue Engineering,” Chapter 18, 347-364, Nanobiotechnology II, Wiley-VCH, (Eds. Mirkin, Niemeyer) (2007); Hamerick et al., “Synthesis of Cell Structures,” Chapter 5, 73-101, Nanoscale Technology in Biological Systems, (Eds. Greco et al.) (2005); US Patent Publication 2007/0224175.

### SUMMARY

Provided herein are embodiments including methods of making, compositions, devices, methods of making compositions and devices, methods of using compositions and devices, and kits, among other embodiments.

One embodiment provides a method comprising: providing at least one substrate; providing at least one first tip with at least one first cell-adhesion material disposed thereon, and at least one second tip with at least one second cell-adhesion material disposed thereon, wherein the first cell-adhesion material is different from the second cell-adhesion material; depositing the first cell-adhesion material from the first tip to the substrate to form at least one first deposit, and depositing the second cell-adhesion material from the second tip to the substrate to form at least one second deposit; and wherein the first and second deposits are capable of providing selective binding to at least one first cell so that the first cell selectively binds to the first deposit, and wherein the second deposit is capable of binding to at least one second cell.

In one embodiment, the selective binding provides that a percentage of first deposit is bound to the first cell and not the second cell, and at least 60% of the first deposit binds to the first cell and not the second cell. In one embodiment, the selective binding provides that a percentage of first deposit is bound to the first cell and not the second cell, and at least 70% of the first deposit binds to the first cell and not the second cell. In one embodiment, the selective binding provides that a percentage of first deposit is bound to the first cell and not the second cell, and at least 80% of the first deposit binds to the first cell and not the second cell. In one embodiment, the selective binding provides that a percentage of first deposit is bound to the first cell and not the second cell, and at least 90% of the first deposit binds to the first cell and not the second cell.

In one embodiment, the selective binding provides that a percentage of first deposit is bound to the second cell and not the first cell, and less than 30% of the first deposit binds to the second cell and not the first cell. In one embodiment, the selective binding provides that a percentage of first deposit is bound to the second cell and not the first cell, and less than 20% of the first deposit binds to the second cell and not the first cell.

In one embodiment, the selective binding provides that a percentage of first deposit is bound to the second cell and not the first cell, and less than 10% of the first deposit binds to the second cell and not the first cell.

In one embodiment, the selective binding provides that a percentage of first deposit is bound to the first cell and not the second cell, and at least 70% of the first deposit binds to the first cell and not the second cell, and wherein selective binding provides that a percentage of first deposit is bound to the second cell and not the first cell, and less than 20% of the first deposit binds to the second cell and not the first cell.

In one embodiment, the first deposit and the second deposit on the substrate provide an array, wherein at least 70% of the deposits on the array have cells binding to the array.

In one embodiment, the first deposit and the second deposit on the substrate provide an array, wherein at least 70% of the deposits on the array have cells binding to the array, and at least 80% of the cells are single cells.

In one embodiment, the substrate comprises a surface layer adapted to covalently bind to the first and/or second cell adhesion material. In one embodiment, the substrate is an epoxy-coated glass.

In one embodiment, the first tip and the second tip are nanoscopic tips. In one embodiment, the first tip and the second tip do not comprise a surface coating. In one embodiment, the first tip and second tip each have a tip radius of about 100 nm or less. In one embodiment, the first tip and second tip each have a tip radius of about 50 nm or less. In one embodiment, the first tip and the second tip are solid tips without a hole or channel in the tip. In one embodiment, the first tip and the second tip are each disposed on a cantilever. In one embodiment, the first tip and the second tip are each disposed on an array of tips which is cantilever free. In one embodiment, the first tip and the second tip are each disposed on an array of tips, wherein the tips are disposed on cantilevers.

In one embodiment, the first and second cell-adhesion materials each comprise at least one protein or peptide. In one embodiment, the first cell adhesion material and the second cell adhesion material are each disposed on the tips from an ink solution. In one embodiment, the first and second cell-adhesion materials each comprise at least one extracellular matrix component. In one embodiment, the first cell-adhesion material or second cell-adhesion material comprises fibronectin. In one embodiment, the first or second cell-adhesion material comprises laminin. In one embodiment, the first cell-adhesion material or the second cell-adhesion material comprise a cell binding ligand. In one embodiment, the first cell-adhesion material or the second cell-adhesion material comprise single stranded DNA.

In one embodiment, the deposition is carried out at relative humidity of at least 20%. In one embodiment, the deposition is carried out with the tips and substrates moving laterally during deposition to form lines. In one embodiment, the deposition is carried out with the tips and substrate not moving laterally during deposition so that the first and second deposits are dots.

In one embodiment, a plurality of the first deposits are deposited in dot form to form a larger first pattern, and a plurality of the second deposits are deposited in dot form to form a larger second pattern, and the larger first and second patterns are each capable of binding one to five cells. In one embodiment, a plurality of the first deposits are deposited in dot form to form a larger first pattern, and a plurality of the second deposits are deposited in dot form to form a larger second pattern, and the larger first and second patterns are each capable of binding only one cell.

In one embodiment, the first deposit and the second deposit each comprise a dot, wherein each dot has a lateral dimension of 30 μm or less. In one embodiment, the first deposit and the second deposit each comprise a dot, wherein each dot has a lateral dimension of 15 μm or less. In one embodiment, the first deposit and the second deposit each have a lateral dimension of 60 μm or less. In one embodiment, the first deposit and the second deposit each have a lateral dimension of 25 μm or less.

In one embodiment, the embodiment further comprising the step of functionalizing the substrate to block non-specific cell adhesion. In one embodiment, the embodiment further comprises the step of depositing an assay material on the substrate which is adapted to affect or potentially affect cell function. In one embodiment, the embodiment further comprises binding the first cell to the first deposit and binding the second cell to the second deposit. In one embodiment, the embodiment further comprises binding the first cell to the first deposit and binding the second cell to the second deposit, and applying an assay material adapted to affect or potentially affect cell function.

In one embodiment, the embodiment further comprises binding the first cell to the first deposit and binding the second cell to the second deposit, applying an assay material adapted to affect or potentially affect cell function, and monitoring at least one function of the first cell and the second cell.

In one embodiment, the first deposit and the second deposit are dots and are spaced by a distance of 60 μm or less as measured from the centers of the dots. In one embodiment, the first deposit and the second deposit are spaced by a distance of 30 μm or less as measured from the centers of the dots.

In one embodiment, a plurality of first deposits are deposited to form a larger first pattern and a plurality of second deposits are deposited to form a larger second pattern, and the first and second patterns are spaced by a distance that allows a direct interaction between the first cell and the second cell. In one embodiment, a plurality of first deposits are deposited to form a larger first pattern and a plurality of second deposits are deposited to form a larger second pattern, and the first and second patterns are each of an arbitrary shape for controlling cell shape.

In one embodiment, the first cell and/or second cell is a stem cell or a progenitor cell. In one embodiment, only one of the first cell or the second cell is a stem cell or a progenitor cell, and the other of the first cell or second cell is not a stem cell or a progenitor cell.

In one embodiment, deposition is carried out over a cross sectional area of at least 1 cm². In one embodiment, deposition is carried out over a cross sectional area for attaching at least 100 pairs of interacting cells.

Another embodiment provides a method comprising: depositing at least one cell adhesion material from at least one tip onto at least one substrate, co-culturing at least two different cells on the substrate, wherein at least one of cells binds to the deposited cell adhesion material.

In one embodiment, the tip is a nanoscopic tip. In one embodiment, the tip has a tip radius of about 100 nm or less. In one embodiment, the tip has a tip radius of about 50 nm or less.

In one embodiment, the cell adhesion material is a protein or peptide. In one embodiment, the depositing of the cell adhesion material form a deposit have a lateral dimension of about 10 microns or less. In one embodiment, the depositing step is carried out a plurality of times to form a pattern, and the pattern binds to only one cell. In one embodiment, the depositing step is carried out a plurality of times to form a pattern, and the pattern binds to only one to five cells. In one embodiment, the depositing step is carried out a plurality of times in the form of dot depositions, and the depositions are spaced within 60 microns of each other as measured from the center of the dots. In one embodiment, the depositing step is carried out a plurality of times in the form of dot depositions, and the depositions are spaced within 30 microns of each other as measured from the center of the dots.

Another embodiment provides an article comprising: at least one substrate; at least one first deposit deposited on the substrate, wherein the first deposit comprises at least one first cell-adhesion material capable of binding to at least one first cell; at least one second deposit deposited on the substrate, wherein the second deposit comprises at least one second cell-adhesion material different from the first cell-adhesion material, wherein the first and second deposits are capable of providing selective binding to at least one first cell so that the first cell selectively binds to the first deposit, and wherein the second deposit is capable of binding to at least one second cell, and wherein the first deposit and the second deposit each have at least one lateral dimension of 60 μm or less.

In one embodiment, the substrate is an epoxy-coated glass.

In one embodiment, the article comprises a plurality of first deposits which form a first pattern, and the article comprises a plurality of second deposits which form a second pattern, and the first and second patterns are each capable of binding one to five cells. In one embodiment, the article comprises a plurality of first deposits which form a first pattern, and the article comprises a plurality of second deposits which form a second pattern, and the first and second patterns are each capable of binding one cell.

In one embodiment, the first deposit and the second deposit each comprise a dot, and wherein each dot has a lateral dimension of 30 μm or less. In one embodiment, the first deposit and the second deposit each comprise a dot, and wherein each dot has a lateral dimension of 15 μm or less. In one embodiment, the first deposit and the second deposit each have a lateral dimension of 50 μm or less.

In one embodiment, the cell-adhesion material comprises at least one protein or peptide. In one embodiment, the cell-adhesion material comprises at least one cell receptor. In one embodiment, the cell-adhesion material comprises at least one extracellular matrix component. In one embodiment, the cell-adhesion material comprises at least single stranded DNA. In one embodiment, the cell adhesion material comprises at least one cell binding ligand. In one embodiment, the first cell-adhesion material comprises fibronectin. In one embodiment, the second cell-adhesion material comprises laminin.

In one embodiment, the embodiment further comprises an assay material deposited on the substrate, wherein the assay material is adapted to affect or potentially affect cell function.

In one embodiment, the first deposit and the second deposit are dots, and are spaced by a distance of 60 μm or less measured from the center of the dots. In one embodiment, the first deposit and the second deposit are dots, and are spaced by a distance of 30 μm or less measured from the center of the dots. In one embodiment, the first deposit and the second deposit are spaced by a distance that allows a direct interaction between the first cell and the second cell. In one embodiment, the first pattern and the second pattern are each of an arbitrary shape for controlling cell shape. In one embodiment, the article further comprising first and second cells disposed on the cell adhesion materials.

Another embodiment provides a microarray comprising: at least one substrate, at least one first deposit comprising at least one first cell-adhesion material deposited on the substrate and at least one first cell bound to the first cell-adhesion material; at least one second deposit comprising at least one second cell-adhesion material deposited on the substrate and at least one second cell bound to the second cell-adhesion material; wherein the first deposit and the second deposit each has at least one lateral dimension of 60 μm or less; wherein the substrate is blocked in areas not occupied by the first cell-adhesion material and the second cell-adhesion material to prevent non-specific cell binding.

In one embodiment, a plurality of first deposits and a plurality of second deposits bind to one to five cells.

In one embodiment, the first deposit and the second deposit each are dots, and wherein each dot has a lateral dimension of 30 μm or less.

In one embodiment, the cell-adhesion material comprises at least one protein or peptide.

In one embodiment, the embodiment further comprises at least one assay material deposited on the substrate and contacting the first cell, wherein the assay material is adapted to affect or potentially affect cell function. In one embodiment, the embodiment further comprises at least one assay material deposited on the substrate and contacting the second cell, wherein the assay material is adapted to affect or potentially affect cell function.

In one embodiment, the first deposit and the second deposit are spaced by a distance of 60 μm or less.

In one embodiment, the first cell contacts the second cell.

In one embodiment, the first cell directly or indirectly interacts with the second cell.

In one embodiment, the first or second cell is a stem cell.

Another embodiment provides a method for co-culturing cells, comprising: providing at least one patterned substrate comprising at least one first pattern and at least one second pattern, the first pattern comprising at least one deposited first cell-adhesion material in at least one first deposit capable of binding to at least one first cell, the second pattern comprising at least one deposited second cell-adhesion material capable of binding to at least one second cell, but wherein the first and second deposits are capable of providing selective binding to the at least one first cell so that the first cell selectively binds to the first deposit, selectively binding the first cell to the first pattern on the patterned substrate; binding the second cell to the second pattern on the patterned substrate; and monitoring the interaction between the first cell bound to the first pattern and the second cell bound to the second pattern.

In one embodiment, the first cell is a stem cell. In one embodiment, the second cell is a stem cell.

In one embodiment, the first pattern and the second pattern each has at least one lateral dimension of between 10 to 60 microns.

In one embodiment, the first pattern and the second pattern each comprises a plurality of deposited dots, and wherein each dot has a lateral dimension of 10 μm or less.

In one embodiment, the cell-adhesion material comprises at least one protein or peptide.

In one embodiment, the first pattern and the second pattern are spaced by a distance of between 30 to 60 microns.

In one embodiment, the first cell contacts the second cell.

In one embodiment, the first cell directly or indirectly interacts with the second cell.

In one embodiment, the patterned substrate is blocked in areas not occupied by the first cell-adhesion material and the second cell-adhesion material to prevent non-specific cell binding.

At least one advantage for one embodiment is the ability to precisely control dimensionally the co-culturing at high resolution and accuracy.

At least one additional advantage for at least one embodiment is single cell control of multiple cell types on a printed substrate.

At least one additional advantage for at least one embodiment includes that patterning of subcellular-sized features allows for better control of cell function compared to prior art.

At least one additional advantage for at least one embodiment includes the ability to modify the pattern as easily and even while an experiment is in progress.

At least one additional advantage for at least one embodiment includes that two or more proteins can be patterned easily without mask alignment issues

At least one additional advantage for at least one embodiment includes precise placement of interacting cells to enable automation of readout methods.

At least one additional advantage for at least one embodiment is the ability to isolate cells from a population thereby investigating the interaction between two cells without signaling from other neighboring cells.

At least one additional advantage for at least one embodiment is the ability to follow the interaction over time since the method could be non-destructive.

### BRIEF DESCRIPTION OF FIGURES

FIGS. 1A-C illustrate an embodiment for tip-based patterning of cell-adhesion materials.

FIGS. 2A-C illustrate an embodiment for cells bound to the patterned cell-adhesion material, including the morphology and number of the cells.

FIGS. 3A-H illustrate an embodiment for the co-culturing of 3T3 fibroblasts with C2C12 myoblasts.

### DETAILED DESCRIPTION

### Introduction

All references cited herein are incorporated by reference in their entirety.

Priority U.S. provisional application Ser. No. 61/491,795 filed May 31, 2011 is hereby incorporated by reference in its entirety including, for example, working examples, figures, and claims.

Patterning and use of patterning in biological applications are described in, for example, WO 2008/141,048; WO 2010/007,524; WO 2010/047,939; WO 2011/014,845; and 2011/008,781.

### Substrate

Substrates known in the art for biological arrays can be used. Substrates can be rigid or flexible. They can be flat or they can have depressions, grooves, wells, protrusions, or other surface physical features. They can comprise glasses or plastics. They can be membranes.

One preferred example is a glass substrate including high quality low fluorescence glass. Embodiments include a glass slide or a glass cover slip. Patterning can be carried out edge-to-edge if desired. Patterning can be carried out over an entire glass slide.

The substrate can be surface treated if desired to facilitate deposition. The substrate can be cleaned. The substrate can be treated to be hydrophilic or hydrophobic. In one embodiment, the substrate is an epoxy-coated glass.

The substrate can be also called a chip. The chip can be rectangular or square. The length and width can be, for example, 1 mm to 100 mm or 5 mm to 50 mm.

The substrate thickness can be, for example, 50 microns to 500 microns, or about 100 microns to about 250 microns.

Substrates used in nanolithography can be used including, for example, substrates described in U.S. Pat. Nos. 6,635,311; 6,827,979; and 7,744,963 (Mirkin et al.).

Substrates can be modified with surface treatments including treatments relevant to cellular adhesion and the blocking of cellular adhesion. See, for example, U.S. Pat. No. 7,695,967.

Substrates can be marked to show addressable sites. The substrate can show grids, horizontal lines, vertical lines, indicia and markings, and other identification features.

In one embodiment, the substrate comprises a surface layer adapted to covalently bind to the first and/or second cell adhesion material.

In one embodiment, the substrate is an epoxy-coated glass.

### Tips and Nanoscopic Tips

Tips used for deposition, including nanoscopic tips, are known in the art. They can be, for example, scanning probe microscope tips, including atomic force microscope tips. In one embodiment, the nanoscopic tips are disposed at the end of cantilevers. In another embodiment, the nanoscopic tips are free of cantilevers, including polymer pens described in Huo et al., Science, 321: 1658-1660 (2008). The nanoscopic tips can be solid and non-hollow. They can be free of an aperture. They can have a tip radius of less than 100 nm, for example, or less than 50 nm, or less than 25 nm, for example. The nanoscopic tips can be sharpened and cleaned by methods known in the art. If desired, they can be surface treated to improve deposition as known in the art. See, for example, US patent publication 2008/0269073 (nucleic acid arrays), US patent publication 2003/0068446 (protein arrays), and US patent publication 2002/0063212 (DPN). Plasma cleaning can be used as needed. In one embodiment, the nanoscopic tips are NanoInk M-type tips. See, for example, U.S. patent application Ser. No. 13/064,766 (NanoInk, Inc., Fragala et al.; published: 2011/0274839).

A plurality of nanoscopic tips can be used together in a pen array for depositing the cell-adhesion material. Pen arrays are known in the art. See, for example, US patent publication 2008/0105042. The pen array can be either a one-dimensional array or a two-dimensional array. In one embodiment, the pen array comprises a plurality of cantilevers each comprising a tip. The number of cantilevers in such a pen array can be, for example, at least 4, at least 8, at least 12, or at least 250.

A first tip can be used; a second tip can be used. The first tip and the second tip can be different tips.

### Cell-Adhesion Material

Cell adhesion materials are known in the art. In one embodiment, cell adhesion materials to be patterned on the substrate include, for example, extracellular matrix (ECM) proteins, such as Fibronectin, Laminin, Tenacin-C, Collagens I, II, IV, Matrigel, Vitronectin, and the like. Other examples include cell receptors, growth factors, cytokines, and other signaling proteins and peptides. Further examples include aptamers, antibodies, carbhohydrates, lipids, and polymers that preferentially bind to cells under certain conditions. Other embodiments include (i) cell binding ligands and attachment of cells overexpressing the receptors to these ligands, and (ii) single stranded DNA and attachment of cells with complementary DNA on the cell surface. Examples of cell binding ligands include growth factors, integrin binding proteins, and antibodies.

For example, fibronectin micropatterns are described in Kwon et al., Genes & Development, 2008 (“Mechanisms to Suppress Multipolar Divisions in Cancer Cells with Extra Centrosomes”). Laminin micropatterns are described in Turcu et al., J. Neurosci Methods, 131(1-2):141-148 (2003). Extracellular matrix patterned by microcontact printing is described in Thery, et al., Nature Cell Biology, 7, 10, 947-953, 2005 (“The Extracellular Matrix Guides the Orientation of the Cell Division Axis”). Adhesive micropatterns are also described in Thery et al., Nature, 1-5, 2007 (“Experimental and Theoretical Study of Mitotic Spindle Orientation”). Cell adhesion materials are also described in, for example, M. C. Beckerle (Ed.), Cell Adhesion, 2001. Cell adhesion motifs such as, for example, RGD are known in the art.

The cell-adhesion material may be one component of an ink adapted for deposition onto a substrate from a nanoscopic tip. The ink may comprise more than one cell adhesion material. The ink may comprise at least one solvent. The ink may comprise at least one carrier and/or at least one additive to facilitating effective deposition. Methods and/or compositions for facilitating protein/peptide deposition have been described in, for example, US 2009/0143246, US 2010/0048427, and U.S. application Ser. No. 12/140,780 (PCT/US2008/067231) (Mirkin et al.). In one embodiment, the viscosity, surface tension, and hydrophilicity of the ink can be controlled.

### Depositing Cell-Adhesion Material

Deposition methods are known in the art including direct write deposition and nanolithography methods. Direct write methods are described in, for example, Pique, Chrisey (Eds.), Direct-Write Technologies for Rapid Prototyping Applications, 2002. Examples include ink jet printing (Chapter 7), micropen printing (Chapter 8), thermal spraying (Chapter 9), Dip-Pen Nanolithographic printing (Chapter 10), electron beam lithography (Chapter 11), focused ion beam (Chapter 12), laser-related methods including micromachining (Chapters 13-17),

The deposited material can form a deposit or deposition shape. One or more deposition shapes can further form a pattern. The shapes and patterns can be repeated across the substrate surface. The size, shape, and chemical functionality of the deposition shape and pattern can be adapted to control binding. See, for example, U.S. Pat. No. 7,569,340.

One deposition example is use of a tip which comprises a material to be deposited on the end of the tip, and transferring the material from the tip to the substrate. If the tip is held stationary with respect to the substrate, the deposition can result in a dot or disc formation. For example, the dot or disc can be characterized by a diameter. If the tip is moved with respect to the substrate, a line or curvilinear feature can be prepared. The line can be formed into a larger pattern such as a square or rectangle. In addition, a series of dots can be also patterned into a square, rectangle, or triangle. Other shapes can include, for example, crossbows, H's, Y's, L's, or any other arbitrary shapes.

Nanolithography methods can be used including, for example, methods described in U.S. Pat. Nos. 6,635,311; 6,827,979; and 7,744,963 (Mirkin et al.). Additional methods are described in, for example, U.S. Pat. No. 7,344,756, WO 2010/096593, and WO 2009/132,321 (Mirkin et al.). Furthermore, patterning devices, including tips and cantilevers and associated methods, are described in, for example, U.S. provisional application 61/324,167 filed Apr. 14, 2010. Protein arrays can be prepared by deposition methods as described in, for example, Mirkin et al., “PEPTIDE AND PROTEIN ARRAYS AND DIRECT-WRITE LITHOGRAPHIC PRINTING OF PEPTIDES AND PROTEINS,” US Patent Publication 2005/0009206; and Mirkin et al., “PEPTIDE AND PROTEIN NANOARRAYS,” US Patent Publication 2003/0068446.

Deposition can be carried out with use of instruments, devices, and consumables provided by NanoInk (Skokie, Ill.) including, for example, the NLP 2000 and DPN 5000 instruments. Other products include pens and pen arrays, chips, substrates, and inkwells.

The deposition can produce dots or lines on the substrate. A series of dots can be formed which are arranged in linear manner.

The size of the shapes and patterns can be adapted to conform to the application and the size of the cell. Physical changes, as well as chemical changes, on the cell can be determined.

Deposition can be carried out so the individual pattern comprises, for example, 1 to 5,000 dots, or 1 to 1,000 dots, or 1 to 100 dots, or 1 to 10 dots. The deposition can be carried out so the substrate comprises, for example, one or more, ten or more, 50 or more, or 100 or more patterns. No fixed upper limit is present but the substrate can comprise less than 5,000 individual patterns, or less than 1,000, or less than 100 individual patterns.

The temperature and humidity for dip-pen nanolithography are known in the art. In one embodiment, the cell-adhesion material is deposited in general room temperature (22-25° C.) and 20%-40% relative humidity. If the relative humidity is under 20% or over 50%, an environmental chamber is recommended for controlling the temperature and humidity levels. If the humidity is too low, the deposited material could dry and may lose their functionality. Environmental control devices for patterning are described in, for example, WO 2010/102,231.

In one embodiment, the deposition of cell-adhesion material does not involve photolithography. In another embodiment, the deposition of cell-adhesion material does not involve inkjet printing. In another embodiment, the deposition of cell-adhesion material does not involve microcontact printing. In a further embodiment, the deposition of cell-adhesion material do not involve the use of a polymer stamp.

### Deposits and Patterns

Deposits can comprise cell-adhesion materials deposited from the tips to the substrate.

In one embodiment, at least one first deposit and at least one second deposit are fabricated onto the substrate. The first deposit comprises a first cell-adhesion material capable of binding to a first cell but not a second cell, whereas the second deposit comprises a second cell-adhesion material capable of binding to the second cell.

The first deposit and the second deposit can each have a lateral dimension of, for example, 200 μm or less, or 100 μm or less, or 50 μm or less, or 20 μm or less, or 10 μm, or one micron or less.

The first pattern and the second pattern can comprises a plurality of dots each having a lateral dimension of, for example, about 50 μm or less, or about 10 μm or less, or about 1 μm or less, or about 100 nm or less.

The first pattern and the second pattern can each bind to, for example, about 1 to 50 cells, or about 1 to 10 cells, or about 1 to 5 cells, or about 1 cell on average.

The first pattern and the second pattern can be spaced by a distance of, for example, 200 μm or less, or 100 μm or less, or 50 μm or less, or 20 μm or less, or 10 μm or less. The first pattern and the second pattern can each bind to a different cell, and be spaced by a distance that allows the two cells to contact each other, or directly or indirectly interact with each other.

The first pattern and the second pattern can be of arbitrary shapes that are the adapted to control shape of the cells bound to the patterns.

In one embodiment, the present invention can precisely control the size, shape, and position of the first pattern and the second pattern so that two different cell types can be placed side-by-side, within microns of each other, on the same substrate.

In one embodiment for single cell binding, either a single spots of 10-14 micron diameter or a 2×2 pattern of spots of 6-8 micron diameter and 15 micron pitch can be used. The spacing between the first and second pattern for cell-cell interaction and optimal binding can be between 30-60 um. The number of cells bound per pattern depends on the size of the pattern, which can be determined by the user of the method.

### Capable of Binding and Selective Binding

In one embodiment, the first deposit is capable of binding to at least one first cell, and wherein the second deposit is capable of binding to at least one second cell but selective binding is carried out. Hence, some selectivity in cell binding is important. The selectivity does not have to provide absolute selectivity, but the selectivity should be high enough to allow for a useful result.

For example, in one embodiment, when applying a sufficient number of first cells to a substrate patterned with both the first cell-adhesion material and the second cell-adhesion material, at least 70% of the patterns of the first cell-adhesion material have the first cell attached; however, due to the selectively or discrimination of the second cell-adhesion material against the first cell, less then 20% of the patterns of the second cell-adhesion material have the first cell attached. In another embodiment, more than 80% of the patterns of the first cell-adhesion material have the first cell attached. In another embodiment, less than 5% of the patterns of the second cell-adhesion material have the first cell attached.

In comparison, in one embodiment, when applying a sufficient number of second cells to the substrate patterned with both the first cell-adhesion material and the second cell-adhesion material, at least 50% of the patterns of the second cell-adhesion material have the second cell attached. In another embodiment, more than 70% of the patterns of the second cell-adhesion material have the second cell attached. In another embodiment, more than 80% of the patterns of the second cell-adhesion material have the second cell attached.

### First Cell and Second Cell

Cells can bind to the patterned substrate via the cell-adhesion materials. A wide variety of cells are known and can be used. See, for example, Pollard and Earnshaw, Cell Biology, 2^{nd} Ed., 2008. Stem cells can be used. See, for example, Lanza (Ed.), Essentials of Stem Cell Biology, 2006.

The cell can be, for example, prokaryotic and eukaryotic cells, normal and transformed cell lines, cells from transgenic animals, transduced cells, neoplastic cells, cells with reporter genes or other biochemical reporters, cells associated with any disease, and cultured cells, which may be derived from animal, bacteria, plant, fungus, viruses, prions, or with respect to tissue origin, heart, lung, liver, brain, vascular, lymph node, spleen, pancreas, thyroid, esophageal, intestine, stomach, thymus, malignancy, atheroma, pathological lesion, and the like.

Two different cells can bind to different cell-adhesion material patterned on the substrate, and be positioned side-by-side, within microns of each other. The two cells may contact each other, or directly or indirectly interact with each other. In a preferred embodiment, only one single cell is interacting with another single cell.

In one embodiment, the first cell is a stem cell, such as an endothelial progenitor cells or a bone marrow-derived mesenchymal stem cell, and the second cell is a cardiac myocytes. In another embodiment, the first cell is 3T3 fibroblast, the second cell is C2C12 myoblast.

In one embodiment, “map” cell placement is carried out, while cell shape, cluster size, and cell-cell spacing can be precisely controlled. With the precision of cell mapping, imaging artifacts can be eliminated, while investigator-introduced variables can be largely reduced.

The size of the cells can be, for example, between 1 and 200 microns, or 4 and 135 microns, or 5 and 50 microns, or 6 and 20 microns, or 7 to 15 microns, or 8-12 microns.

The shape of the cells can be, for example, rounded, cylindrical, star, irregular, and the like.

In one embodiment, cells that can bind to the patterned substrate include primary cell (e.g. Cardiac, liver, neural), stem cell (bone marrow derived MSC, ESC, iPS), and disease state models (Cardiac Fibrosis—Primary heart and fibroblasts).

### Assay Material Adapted to Affect or Potentially Affect Cell Function

Many assay materials can be tested for their effect or potential effect on cell function. Examples include drug molecules, toxins, nanomaterials, nanoparticles, nanotubes, carbon nanotubes, proteins, and the like.

The assay material can be, for example, any drug or material, which can be put in, for example, a hydrogel and release to the bound cells. Testing multiple drugs/materials on a single piece of glass can be carried out.

The assay material can be, for example, cytokines, chemokines, differentiation factors, growth factors, soluble receptors, prostaglandins, steroids, pharmacologically active drugs, genetically active molecules, chemotherapeutic agents, anti-inflammatory agents, hormones or hormone antagonists, ion channel modifiers, neuroactive agents, toxins, biological and chemical warfare agents, nanoparticles, nanotubes, and any other small proteins or small molecules that affect or potentially affect cellular function.

Assay materials are also described in, for example, US Patent Publication 2004/0248144.

### Monitoring Cell Functions

Techniques for monitoring cell-functions are well known in the art. Cell functions can be monitor in various ways including, for example, cell morphology, cell metabolism, cell division, cell differentiation, gene transcription, and protein expression. See, for example, Alberts et al., Molecular Biology of the Cell, 5^{th} Ed., 2007; Lodis et al., Molecular Cell Biology, 5^{th} Ed., 2007; Pollard and Earnshaw, Cell Biology, 2^{nd} Ed., 2008; and Lanza (Ed.), Essentials of Stem Cell Biology, 2006.

In one embodiment, techniques for monitoring cell-functions include phase microscopy, Live cell fluorescent microscopy, confocal microscopy, total internal reflection fluorescence microscope, and super resolution microscopy. Environmental control for imaging (CO2, humidity, temp) may be necessary. Automated tracking systems and software thereof may be used.

### Substrate Blocking to Prevent Non-Specific Cell Binding

As known among skilled artisans, the substrate surface can be treated to prevent non-specific binding. Known cell blocking agents can be used. Blocking agents can be natural or synthetic. Examples include antiadhesive protein albumin, hydrogels based on hyaluronic acid, poly(hydroxyl ethyl methacrylate), polyvinyl alcohol, polyacrylamide, dextran. Other examples include, for example, PEG, PEO, PEG derivatives, PEO derivatives, pluronics, casein, etc. In particular, PBS solutions can be used. For example, at least one blocking solution can be used to treat the substrate surface after the deposition of the patterns. The blocking solution can be, for example, 1-2% bovine serum albumin in PBS, 5% fetal calf serum in PBS, 10% goat serum in PBS, or any other composition that can block the non-specific binding of cells.

### Cell Microarray

A cell microarray can be fabricated. The cell microarray can comprise at least one substrate, at least one first pattern comprising at least one first cell-adhesion material deposited on the substrate and at least one first cell bound to the first cell-adhesion material, and at least one second pattern comprising at least one second cell-adhesion material deposited on the substrate and at least one second cell bound to the second cell-adhesion material. The first pattern and the second cell pattern can each have at least one lateral dimension of 100 μm or less. And the substrate can be blocked in areas not occupied by the first cell-adhesion material and the second cell-adhesion material to prevent non-specific cell binding.

### Cell Co-Culture

Also provided is a method for co-culturing cells. The method, for example, may comprise the step of (1) providing at least one substrate patterned with at least one first pattern and at least one second pattern, with the first pattern comprising at least one first cell-adhesion material capable of binding to at least one first cell but not to at least one second cell and the second pattern comprising at least one second cell-adhesion material capable of binding to the second cell; (2) binding the first cell to the first pattern on the patterned substrate and binding the second cell to the second pattern on the patterned substrate; and (3) monitoring the interaction between the first cell and the second cell.

The method for co-culturing cells may also include a step of blocking the pattern substrate in areas not occupied by the first cell-adhesion material and the second cell-adhesion material to prevent non-specific cell binding.

In one embodiment, assay materials adapted to affect or potentially affect cell function can be applied to the cells on the substrate. The assay material can be either pre-deposited on the substrate or added after the attachment of the cells to the cell-adhesion materials. The response of the cells to such assay materials can then be monitored according to techniques known in the art.

### Applications

Applications for embodiments described herein includes cell-cell interaction studies, drug screening (e.g. drugs that act on the mechanism of cell-cell interaction), cell communication and signaling, tissue engineering, developing artificial tissue constructs, and the like.

### WORKING EXAMPLES

Additional embodiments are provided in the following working examples.

Using the NLP 2000 (NanoInk, Inc, Skokie, Ill.), sub-cellular patterning of multiple proteins was demonstrated to assess various cell functions on two different cell types. Further pattern size and shape was precisely controlled so that these two different cell types were placed side-by-side, within tens of microns of each other, on the same substrate. Patterning was done over a large area in a short time frame, and the flexibility of the method allows for very complex experiments to be performed with short development time. Further, it was shown that one can fabricate experimental substrates, perform experiments, gather results in a short time-frame, and make necessary experimental adjustments, thus minimizing optimization times. The ability to precisely control the cellular microenvironment was demonstrated, which is useful for many different cell culture and tissue engineering industrial and research applications. In particular, tip-based lithography can be used to pattern cell adhesion materials on functionalized glass substrate, such as epoxy-coated glass.

NIH 3T3 fibroblasts and C2C12 myoblasts were purchased from American Type Culture Collection (Manassas, Va.). For expansion and standard culture, complete culture media (CCM) consisted of DMEM supplemented with 10% fetal bovine serum (Atlanta Biologicals, Lawrenceville, Ga.) and 1% antibiotic/antimycotic. Cells were thawed and passaged at least once prior to use.

### Working Example 1

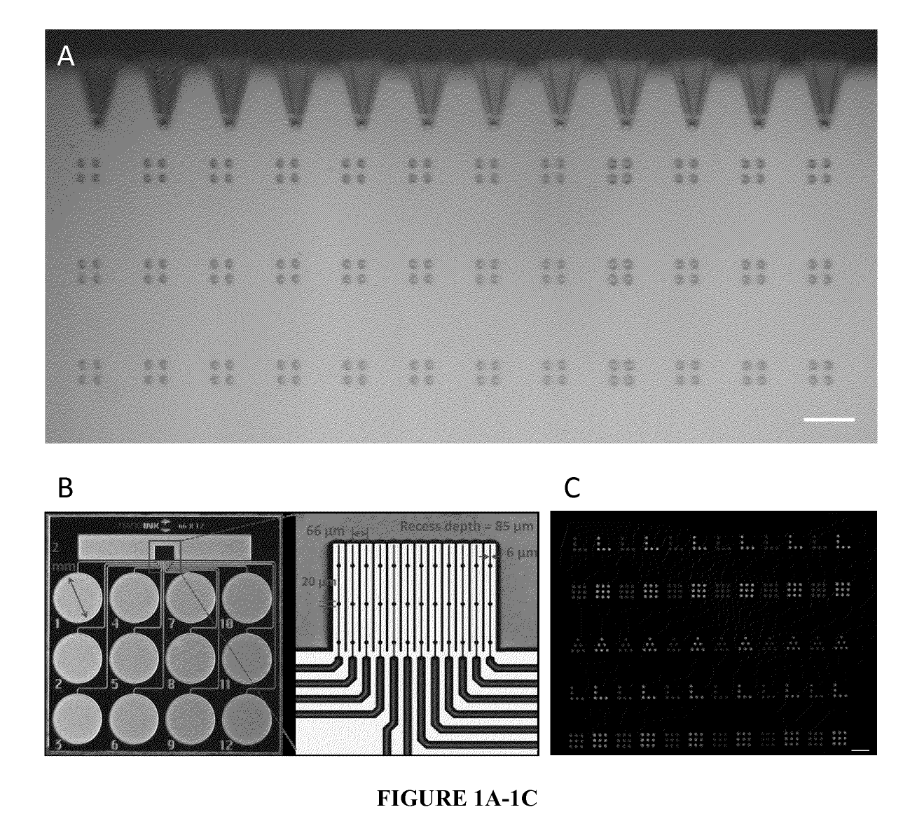

### Preparation of Arrays (FIG. 1)

For example, FIGS. 1A-C show embodiments for tip-based patterning.

(A.) Alternating from left to right, silicon nitride tips transfer both fibronectin and laminin ECM proteins to epoxy coated glass in 2×2 square patterns. Scale bar=50 μm.

(B) Inkwell chip showing individual reservoirs allowing multiplexing of up to 12 different materials. The expanded figure (callout) depicts the microchannels into which silicon nitride tips are dipped to load tips with protein solutions.

(C.) Fibronectin (red) and laminin (green) ECM proteins of different shapes on epoxy functionalized glass surface. Scale bar=50 μm.

FIGS. 1A-C are described more particularly. The NLP 2000 (NanoInk, Inc, Skokie, Ill.) was used for direct deposition of extracellular matrix (ECM) proteins at sub-cellular scales (FIG. 1A). Stock protein solutions of approximately 1 mg/ml were mixed with buffer (NanoInk, Inc, Skokie, Ill.) and loaded into 2 mm diameter individual reservoirs of an inkwell chip (NanoInk, Inc, Skokie, Ill.) (FIG. 1B, left). The reservoirs supply protein solution to microchannels by capillary wicking An array of 12 silicon nitride tips was then dipped into microchannels at the top of the inkwell chip (FIG. 1B, right) allowing individual tips within the 12-tip array to be coated with the same or different protein solution. The tip array was then moved in contact with the glass substrate for transfer of protein solution from the tips to the substrate. Motion was made with nanoscale resolution allowing for precise deposition of various proteins. Patterns of fibronectin and laminin (BD Bioscience, San Jose, Calif.) were fabricated of various sizes and shapes onto epoxy functionalized glass surfaces (Schott North America, Inc., Elmsford, N.Y.) (FIG. 1C).

### Example 2

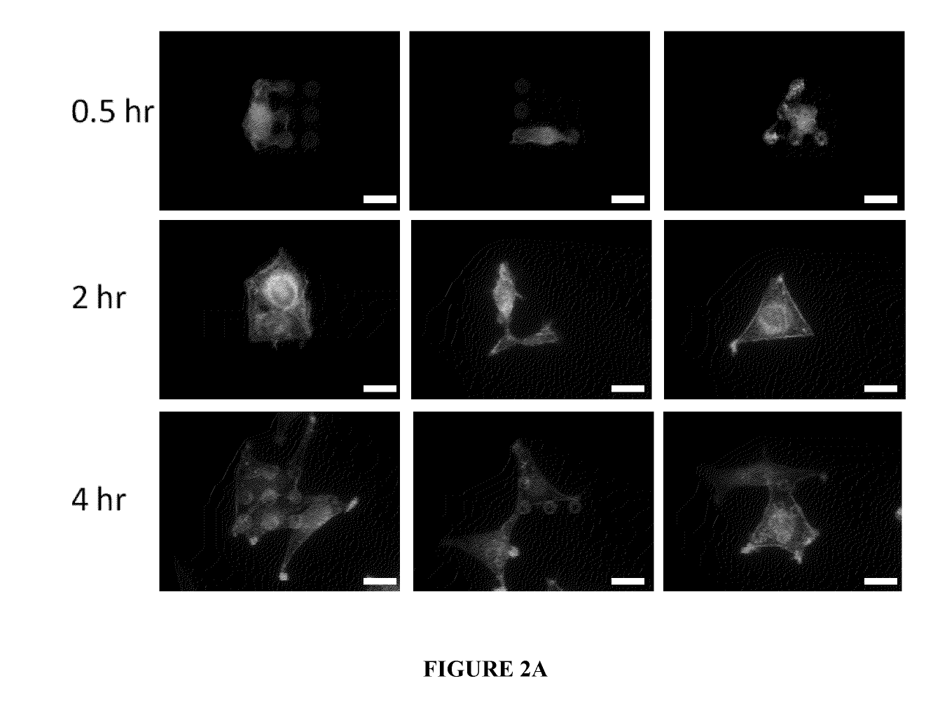

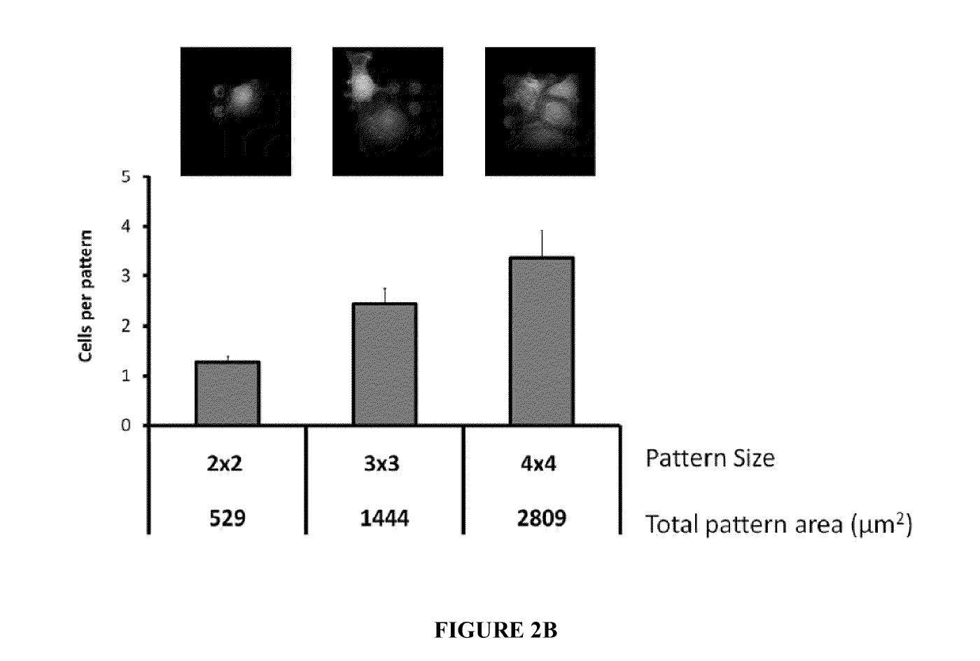

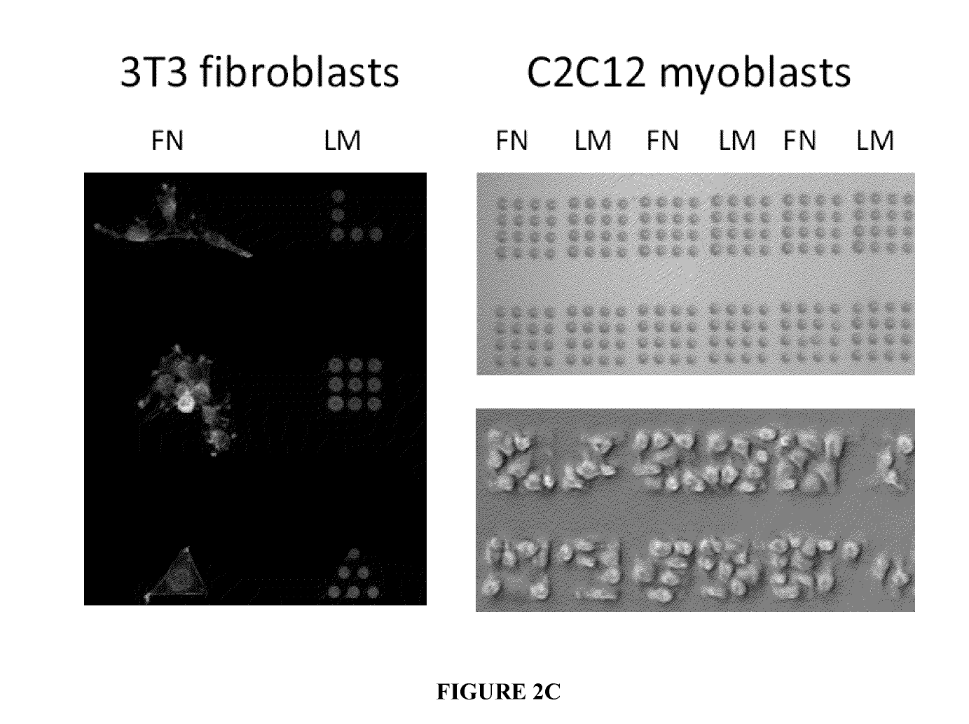

### Cell Binding (FIG. 2)

FIGS. 2A-C show embodiments wherein cell function is determined by pattern shape and composition.

(A.) Cell morphology is controlled by the pattern of fibronectin printed. Cells attach to fibronectin and spread over time. At two hours cell morphology clearly coincides with the protein patterns. Actin filaments become more defined, spreading and migration occurs through 4 hours. Scale bar=20 μm.

(B.) Pattern size determines cluster size. Larger patterns allow more cells to bind and single cell levels can be achieved with 2×2 patterns of fibronectin covering a total area of 529 μm².

(C.) 3T3 fibroblasts preferentially bind to fibronectin versus laminin, and C2C12 myoblasts will attach to both proteins after 30 minutes of culture and washing.

FIGS. 2A-C are described more particularly. In order to determine cell response to various shapes of ECM protein, fibronectin was patterned on the same substrate in square, L and triangle shapes and cell morphology was assess over a period of four hours. After substrate “printing” the protein spots react with the surface for at least 1 hour. Substrates were rinsed briefly with PBS and incubated at room temperature with 2% BSA solution to block non-specific cell binding. After blocking, substrates were washed and 3T3 fibroblasts were added at 1×10⁵ cells/cm² in DMEM supplemented with ITS+1 (Invitrogen, Carlsbad, Calif.). Cells were allowed to attach for 30 minutes at which point non-attached cells were washed away. Cells were fixed with 4% paraformaldehyde at 0.5, 2 or 4 hours. Monoclonal anti-human fibronectin (Sigma, St. Louis, Mo.) labeled pattern spots on the substrate and a secondary antibody allowed visualization using fluorescence microscopy. Phalloidin-488 (Invitrogen) was used to visualize actin filament formation over time. DAPI preservative was used to preserve samples as well as label nuclei. A fluorescent microscope (AxioImager Zlm, Carl Zeiss, Thornwood, N.Y.) was used for imaging.

In order to test the number of cells binding to patterns of various sizes, fibronectin was patterned into square patterns of 2×2, 3×3 or 4×4 matrices. 3T3 fibroblasts were added as described above at a density of 1×10⁵ cells/cm². For differential cellular binding experiments, fibronectin and laminin patterns of square, ‘L’ and triangle shapes were printed side-by-side. 3T3 fibroblasts or C2C12 myoblasts were added as described above. Cells were fixed at 2 hours with 4% paraformaldehyde, labeled and imaged.

For coculture experiments, fibronectin and laminin patterns were printed in various shapes and sizes onto epoxy coated glass as described above. 3T3 fibroblasts and C2C12 myoblasts were labeled separately with Qtracker Qdots (Invitrogen) of 525 or 655 nm excitation, respectively. 3T3 fibroblasts were then added in ITS+1 supplemented DMEM at 1×10⁵ cells/cm² and allowed to attach for 30 minutes. Non-attached cells were washed and phase contrast images collected to illustrate fibroblast attachment. Next, labeled C2C12 myoblasts were added in ITS+1 supplemented DMEM at 1×10⁵ cells/cm² and allowed to attach for 30 minutes before washing of non-attached cells. Phase contrast images were collected to illustrate myoblast attachment. After 1-3 hours more of culture, the cells were fixed with 4% parafomaldehyde. Laminin and fibronectin were labeled as described above and DAPI preservative added.

3T3 fibroblasts adhere to fibronectin patterns consisting of spots ˜6-8 μm diameter, spaced 15 μm apart (FIG. 2A). Non-specific binding to BSA blocked areas is prevented by washing of substrates 30 minutes after initial cell plating. Over a period of 2 hours, cells take on the shape of the square, L or triangle protein patterns. At 4 hours, stress fiber formation is more defined and spreading and migration increases.

For some cell types, cell cluster size and cell density play an important role in cell function. Here, 3T3 cluster size is controlled by the area of pattern provided. The number of cells per pattern increases with larger fibronectin patterns (FIG. 2B). In general, single cells are observed with 2×2 patterns (529 μm²) increasing to almost 4 cells per pattern for 4×4 patterns (2809 μm²). It is important to note that the cluster size for a given pattern size changes depending on the initial cell density (data not shown) especially for low starting cell densities.

After 30 minutes of pre-plating time, washing and 2 hours of culture there is a clear preference for 3T3 fibroblasts to adhere to fibronectin as compared to laminin (FIG. 2C, left). This preference has been reported previously [9]. Further, C2C12 myoblasts do attach to both fibronectin and laminin (FIG. 2C, right).

### Example 3

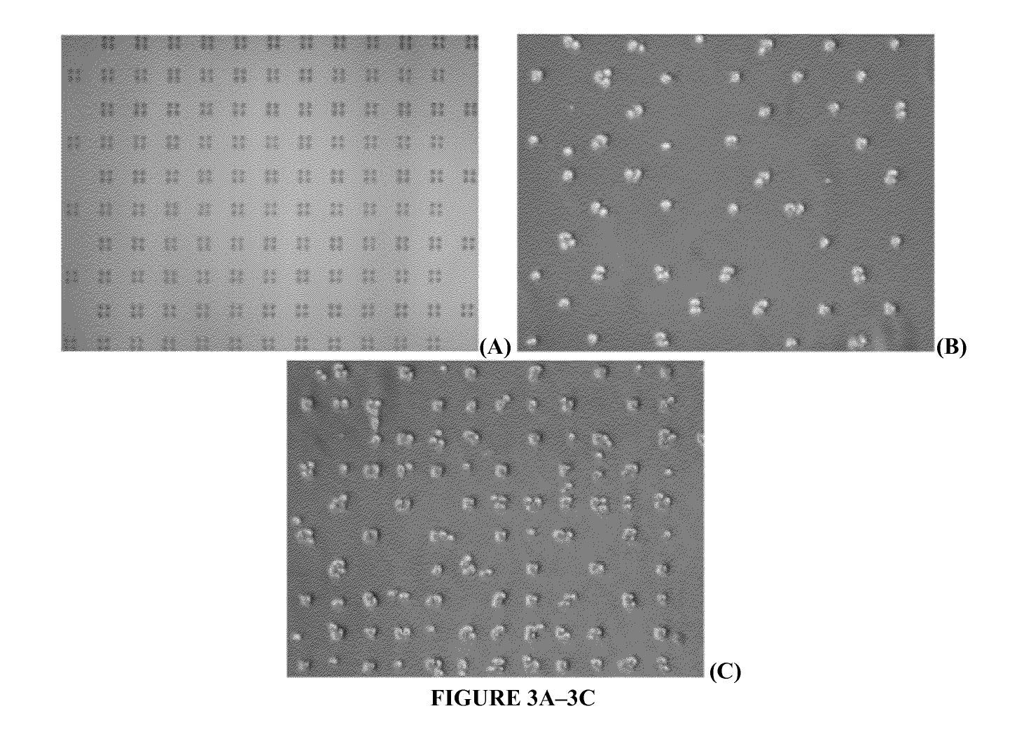

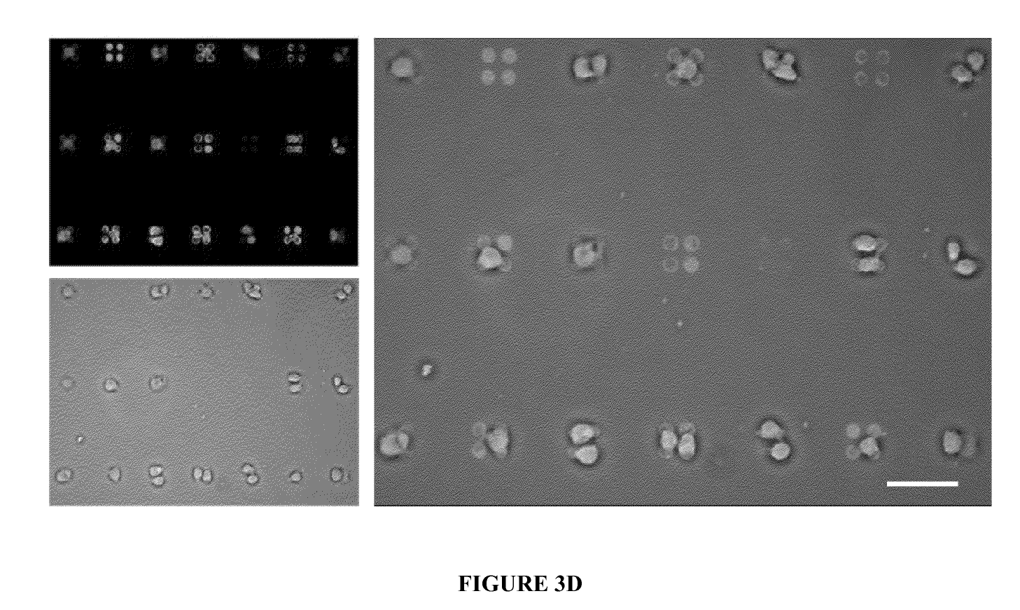

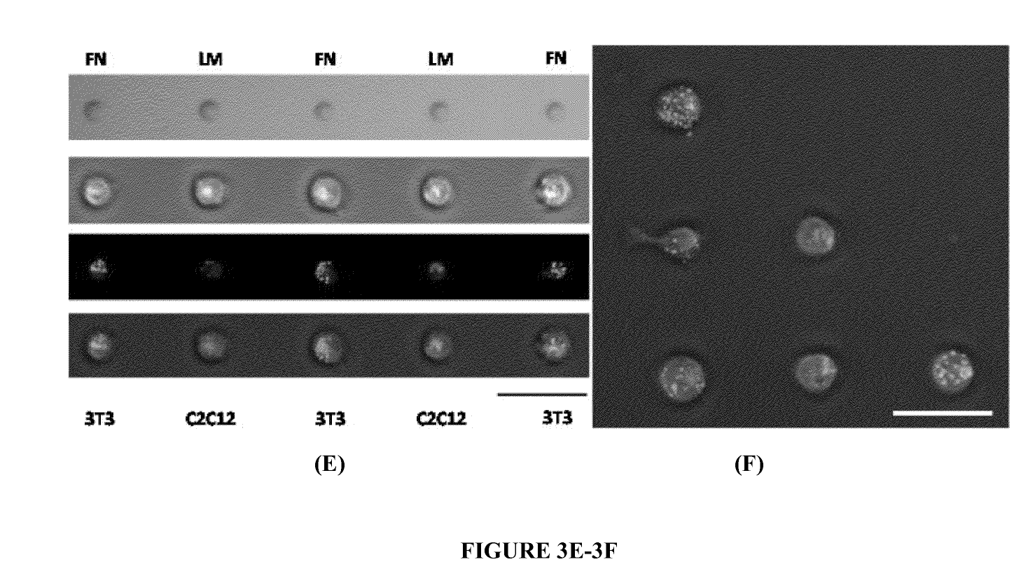

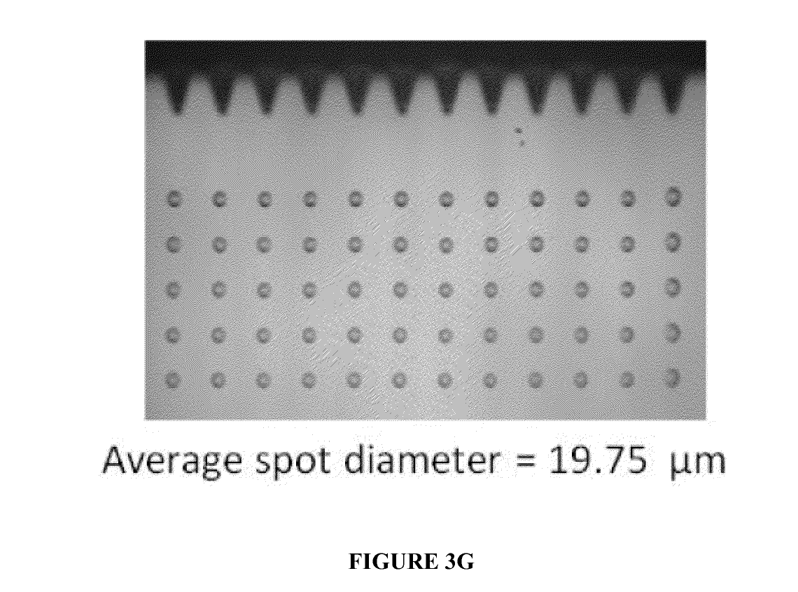

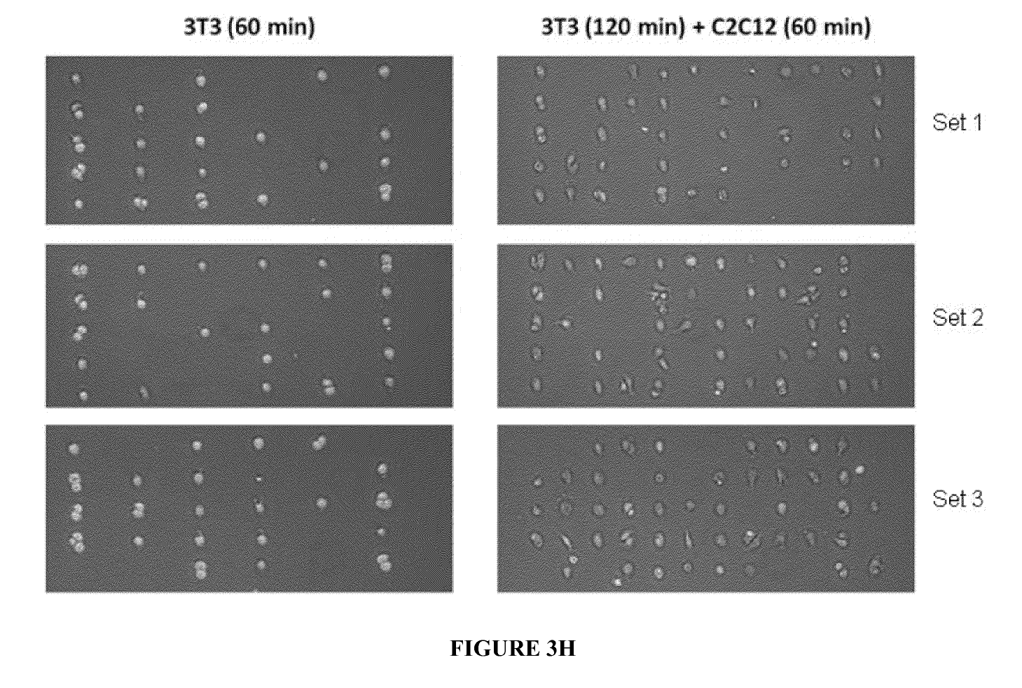

### Co-Culture (FIG. 3)

FIG. 3 shows embodiments comprising the coculture of 3T3 fibroblasts with C2C12 myoblasts.

(A.) Printed array of fibronectin and laminin 2×2 patterns alternating in both the x and y directions.

(B.) Phase contrast image of fibroblasts attached to fibronectin after 30 minutes.

(C.) Phase contrast image of C2C12 myoblasts attached to laminin and in some cases remaining exposed fibronectin patterns. Note images are from the same area of the same substrate at all points. Distance between patterns is 66 μm.

(D.) Fluorescent, phase and combination images show the attachment of 3T3 fibroblast (green Qdots) on fibronectin patterns (red spots) and C2C12 myoblasts (red Qdots) on laminin patterns (green spots).

(E, F.) Single spot printing of diameter between 10-12 μm allows for single cell attachment of 3T3 fibroblasts to fibronectin and C2C12 myoblasts to laminin.

(G.) Relatively large sized single protein spots, about 20 μm diameter allowed for binding of mainly single cells over large areas.

(H.) Images of 3T3 fibroblast attachment and C2C12 myoblast attachment on three 12×5 arrays (30 fibronectin and 30 laminin spots), printed on the same substrate. 3T3 cells were added and 60 minutes later C2C12 cells were added. Statistics of cell attachment on the three sets of patterned substrates are shown in Table 1.

TABLE 1

| | Set 1 | Set 2 | Set 3 | Mean ± SD |
|---|---|---|---|---|
| FN spots with 3T3 attached | 22/30 | 22/30 | 22/30 | 0.73 ± 0.00 |
| LM spots with C2C12 attached | 13/30 | 17/30 | 20/30 | 0.55 ± 0.12 |
| FN spots with C2C12 attached | 4/30 | 5/30 | 5/30 | 0.15 ± 0.02 |
| Total spots with C2C12 attached | 17/30 | 22/30 | 25/30 | 0.71 ± 0.13 |
| Single cells | 32/39 | 35/44 | 37/47 | 0.80 ± 0.02 |

Note:
3 sets - Bleeding dots from same substrate Attachment statistics for both cell types on FN and LM show that approximately 70% of binding areas have cells attached, with 80% of those being single cells.

Additional description is provided for FIGS. 1A-H. Due to the binding characteristics of these two cell types to fibronectin and laminin, co-culture experiments were done demonstrating the ability to precisely control cell placement. Alternating fibronectin and laminin, 2×2 boxes were patterned (FIG. 3A). Qdot labeled 3T3 fibroblasts were then added and allowed to attach (FIG. 3B). Approximately 30 or 60 minutes later, Qdot labeled C2C12 myoblasts were added and allowed to attach for 30 minutes (FIG. 3C). Fluorescent images demonstrate that fibroblasts (green Qdot clusters) bind to fibronectin (red spots) and myoblasts (red Qdot clusters) bind to laminin (green spots) (FIG. 3D). Note that in some instances when fibroblasts did not bind to and cover all exposed fibronectin, that myoblasts were able to attach to fibronectin. This occurred less when larger single spots (10-12 μm diameter) were patterned and single cells attached thus leaving no exposed fibronectin for myoblasts to attach to (FIGS. 3E and 3F). Larger spots of approximately 20 μm diameter (FIG. 3G) also allow for single cell binding. More than 70% of cell binding areas have cell attachment (FIG. 3H and Table 1). Due to the blocking of available fibronectin by attached fibroblasts, the myoblasts attach to laminin with 55% of the printed laminin spots occupied by myoblasts. Further, approximately 80% of the cell binding areas with cell attachment are of single cells.

In summary, embodiments described herein, including FIGS. 1-3, illustrate that a new technique is described to control cell function with precise and known conditions such as cell cluster size, extracellular matrix composition and placement of multiple cells in coculture. Precision co-culture of two cell types, 3T3 fibroblasts and C2C12 myoblasts was demonstrated, allowing the manipulation of cells with sub-cellular precision. Further, it is possible to control the characteristics of one cell type and simulate the environment a second cell type might be exposed to in vivo. Two cell types binding to different ECM proteins was demonstrated, but other cell binding experiments can be carried out. For example, experiments involving printing of cell binding ligands and attachment of cells overexpressing the receptors to these ligands or printing of single stranded DNA and attachment of cells with complementary DNA on the cell surface [10] can be carried out. Importantly, all of the features ‘printed’ can be multiplexed at subcellular scales, allowing for manipulation of single cells.

### CITED REFERENCES

Additional application and teachings are described in the following references, 1-10, cited hereinabove (references 11-14 also provide guidance):
[1] Chen, et al., Geometric control of cell life and death, Science 276 (1997) 1425-1428.
[2] Thery, et al., Micropatterns for Cells: A Microcontact Printing Protocol, Cold Spring Harb. Protoc. 7 (2009) 1-15.
[3] Hui, et al., Constraints on Microcontact Printing Imposed by Stamp Deformation, Langmuir 18 (2002) 1394-1407.
[4] Condorelli, et al., Cardiomyocytes induce endothelial cells to trans-differentiate into cardiac muscle: implications for myocardium regeneration. Proc Natl Acad Sci USA 98 (2001) 10733-10738.
[5] Badorff, et al., Transdifferentiation of blood-derived human adult endothelial progenitor cells into functionally active cardiomyocytes. Circulation 107 (2003) 1024-1032.
[6] Shim, et al.: Ex vivo differentiation of human adult bone marrow stem cells into cardiomyocyte-like cells. Biochem Biophys Res Commun 324 (2004) 481-488.
[7] Li, et al., Bone marrow mesenchymal stem cells differentiate into functional cardiac phenotypes by cardiac microenvironment. J Mol Cell Cardiol 42 (2007) 295-303.
[8] Anversa, et al., Concise review: stem cells, myocardial regeneration, and methodological artifacts. Stem Cells 25 (2007) 589-601.
[9] Chambers, et al., Adhesion of Metastatic, ras-transformed NIH 3T3 Cells to Osteopontin, Fibronectin, and Laminin, Cancer Research 53 (1993) 701-706.
[10] Hsiao, et al., Direct Cell Surface Modification with DNA for the Capture of Primary Cells and the Investigation of Myotube Formation on Defined Patterns, Langmuir 25 (2009) 6985-6991.
[11] Walling, et al., Cellular heterogeneity and live cell arrays, Chem. Soc. Rev., (2011) Advance Article.
[12] Lindström, et al., Overview of single-cell analyses: microdevices and applications, Lab Chip 10 (2010) 3363-3372.
[13] Fukuda, et al., Micropatterned cell co-cultures using layer-by-layer deposition of extracellular matrix components, Biomaterials (2006) 1479-1486.
[14] Hui, et al., Micromechanical control of cell-cell interactions, Proc Natl Acad Sci USA 104 (2007) 5722-5726.

1. A method comprising:
providing at least one substrate;
providing at least one first tip with at least one first cell-adhesion material disposed thereon, and at least one second tip with at least one second cell-adhesion material disposed thereon, wherein the first cell-adhesion material is different from the second cell-adhesion material;
depositing the first cell-adhesion material from the first tip to the substrate to form at least one first deposit, and depositing the second cell-adhesion material from the second tip to the substrate to form at least one second deposit; and
wherein the first and second deposits are capable of providing selective binding to at least one first cell so that the first cell selectively binds to the first deposit, and wherein the second deposit is capable of binding to at least one second cell.

2. The method of claim 1, wherein selective binding provides that a percentage of first deposit is bound to the first cell and not the second cell, and at least 60% of the first deposit binds to the first cell and not the second cell.

3-4. (canceled)

5. The method of claim 1, wherein selective binding provides that a percentage of first deposit is bound to the first cell and not the second cell, and at least 90% of the first deposit binds to the first cell and not the second cell.

6. The method of claim 1, wherein selective binding provides that a percentage of first deposit is bound to the second cell and not the first cell, and less than 30% of the first deposit binds to the second cell and not the first cell.

7-8. (canceled)

9. The method of claim 1, wherein selective binding provides that a percentage of first deposit is bound to the first cell and not the second cell, and at least 70% of the first deposit binds to the first cell and not the second cell, and wherein selective binding provides that a percentage of first deposit is bound to the second cell and not the first cell, and less than 20% of the first deposit binds to the second cell and not the first cell.

10. The method of claim 1, wherein the first deposit and the second deposit on the substrate provide an array, wherein at least 70% of the deposits on the array have cells binding to the array.

11. The method of claim 1, wherein the first deposit and the second deposit on the substrate provide an array, wherein at least 70% of the deposits on the array have cells binding to the array, and at least 80% of the cells are single cells.

12. The method of claim 1, wherein the substrate comprises a surface layer adapted to covalently bind to the first and/or second cell adhesion material.

13. The method of claim 1, wherein the substrate is an epoxy-coated glass.

14. The method of claim 1, wherein the first tip and the second tip are nanoscopic tips.

15. The method of claim 1, wherein the first tip and the second tip do not comprise a surface coating.

16. The method of claim 1, wherein the first tip and second tip each have a tip radius of about 100 nm or less.

17. The method of claim 1, wherein the first tip and second tip each have a tip radius of about 50 nm or less.

18. The method of claim 1, wherein the first tip and the second tip are solid tips without a hole or channel in the tip

19. The method of claim 1, wherein the first tip and the second tip are each disposed on a cantilever.

20. The method of claim 1, wherein the first tip and the second tip are each disposed on an array of tips which is cantilever free.

21. The method of claim 1, wherein the first tip and the second tip are each disposed on an array of tips, wherein the tips are disposed on cantilevers.

22. The method of claim 1, wherein the first and second cell-adhesion materials each comprise at least one protein or peptide.

23. The method of claim 1, wherein the first cell adhesion material and the second cell adhesion material are each disposed on the tips from an ink solution.

24. The method of claim 1, wherein the first and second cell-adhesion materials each comprise at least one extracellular matrix component.

25. The method of claim 1, wherein the first cell-adhesion material or second cell-adhesion material comprises fibronectin.

26. The method of claim 1, wherein the first or second cell-adhesion material comprises laminin.

27. The method of claim 1, wherein the first cell-adhesion material or the second cell-adhesion material comprise a cell binding ligand.

28. The method of claim 1, wherein the first cell-adhesion material or the second cell-adhesion material comprise single stranded DNA.

29. The method of claim 1, wherein the deposition is carried out at a relative humidity of at least 20%.

30. The method of claim 1, wherein the deposition is carried out with the tips and substrates moving laterally during deposition to form lines.

31. The method of claim 1, wherein the deposition is carried out with the tips and substrate not moving laterally during deposition so that the first and second deposits are dots.

32. The method of claim 1, wherein a plurality of the first deposits are deposited in dot form to form a larger first pattern, and a plurality of the second deposits are deposited in dot form to form a larger second pattern, and the larger first and second patterns are each capable of binding one to five cells.

33. The method of claim 1, wherein a plurality of the first deposits are deposited in dot form to form a larger first pattern, and a plurality of the second deposits are deposited in dot form to form a larger second pattern, and the larger first and second patterns are each capable of binding only one cell.

34. The method of claim 1, wherein the first deposit and the second deposit each comprise a dot, wherein each dot has a lateral dimension of 30 μm or less.

35. The method of claim 1, wherein the first deposit and the second deposit each comprise a dot, wherein each dot has a lateral dimension of 15 μm or less.

36. The method of claim 1, wherein the first deposit and the second deposit each have a lateral dimension of 60 μm or less.

37. The method of claim 1, wherein the first deposit and the second deposit each have a lateral dimension of 25 μm or less.

38. The method of claim 1, further comprising the step of functionalizing the substrate to block non-specific cell adhesion.

39. The method of claim 1, further comprising the step of depositing an assay material on the substrate which is adapted to affect or potentially affect cell function.

40. The method of claim 1, further comprising binding the first cell to the first deposit and binding the second cell to the second deposit.

41. The method of claim 1, further comprising binding the first cell to the first deposit and binding the second cell to the second deposit, and applying an assay material adapted to affect or potentially affect cell function.

42. The method of claim 1, further comprising binding the first cell to the first deposit and binding the second cell to the second deposit, applying an assay material adapted to affect or potentially affect cell function, and monitoring at least one function of the first cell and the second cell.

43. The method of claim 1, wherein the first deposit and the second deposit are dots and are spaced by a distance of 60 μm or less as measured from the centers of the dots.

44. The method of claim 1, wherein the first deposit and the second deposit are spaced by a distance of 30 μm or less as measured from the centers of the dots.

45. The method of claim 1, wherein a plurality of first deposits are deposited to form a larger first pattern and a plurality of second deposits are deposited to form a larger second pattern, and the first and second patterns are spaced by a distance that allows a direct interaction between the first cell and the second cell.

46. The method of claim 1, wherein a plurality of first deposits are deposited to form a larger first pattern and a plurality of second deposits are deposited to form a larger second pattern, and the first and second patterns are each of an arbitrary shape for controlling cell shape.

47. The method of claim 1, wherein the first cell and/or second cell is a stem cell or a progenitor cell.

48. The method of claim 1, wherein only one of the first cell or the second cell is a stem cell or a progenitor cell, and the other of the first cell or second cell is not a stem cell or a progenitor cell.

49. The method of claim 1, wherein deposition is carried out over a cross sectional area of at least 1 cm².

50. The method of claim 1, wherein deposition is carried out over a cross sectional area for attaching at least 100 pairs of interacting cells.

51. A method comprising:
depositing at least one cell adhesion material from at least one tip onto at least one substrate,
co-culturing at least two different cells on the substrate, wherein at least one of cells binds to the deposited cell adhesion material.

52-60. (canceled)

61. An article comprising:
at least one substrate;
at least one first deposit deposited on the substrate, wherein the first deposit comprises at least one first cell-adhesion material capable of binding to at least one first cell;
at least one second deposit deposited on the substrate, wherein the second deposit comprises at least one second cell-adhesion material different from the first cell-adhesion material,
wherein the first and second deposits are capable of providing selective binding to at least one first cell so that the first cell selectively binds to the first deposit, and wherein the second deposit is capable of binding to at least one second cell, and
wherein the first deposit and the second deposit each have at least one lateral dimension of 60 μm or less.

62-80. (canceled)

81. A microarray comprising:
at least one substrate,
at least one first deposit comprising at least one first cell-adhesion material deposited on the substrate and at least one first cell bound to the first cell-adhesion material;
at least one second deposit comprising at least one second cell-adhesion material deposited on the substrate and at least one second cell bound to the second cell-adhesion material;
wherein the first deposit and the second deposit each has at least one lateral dimension of 60 μm or less;
wherein the substrate is blocked in areas not occupied by the first cell-adhesion material and the second cell-adhesion material to prevent non-specific cell binding.

82-90. (canceled)

91. A method for co-culturing cells, comprising:
providing at least one patterned substrate comprising at least one first pattern and at least one second pattern, the first pattern comprising at least one deposited first cell-adhesion material in at least one first deposit capable of binding to at least one first cell, the second pattern comprising at least one deposited second cell-adhesion material capable of binding to at least one second cell, but wherein the first and second deposits are capable of providing selective binding to the at least one first cell so that the first cell selectively binds to the first deposit,
selectively binding the first cell to the first pattern on the patterned substrate;
binding the second cell to the second pattern on the patterned substrate; and
monitoring the interaction between the first cell bound to the first pattern and the second cell bound to the second pattern.

92-100. (canceled)
